(19) United States
(12) Patent Application Publication
(10) Pub. No.: US 2018/0374099 A1
(43) Pub. Date: Dec. 27, 2018

(54) BIOMETRIC ANALYSIS OF USERS TO DETERMINE USER LOCATIONS

(71) Applicant: GOOGLE INC., Mountain View, CA (US)

(72) Inventors: Amit Litsur, Portola Valley, CA (US); Yichen Zhao, Santa Clara, CA (US); Timothy Zwiebel, San Jose, CA (US)

(21) Appl. No.: 15/630,413

(22) Filed: Jun. 22, 2017

(51) Int. Cl. G06Q 20/40; G06K 9/00; G06F 17/30; A61B 5/1171

(52) U.S. Cl. CPC G06Q 20/40145; G06K 9/00288; G06K 9/00536; G06K 9/00677; G06T 2207/30232; A61B 5/1176; G06K 9/00255; G06T 2207/30201; G06F 17/30247

(57) ABSTRACT

An account management system identifies a user near a location from among a plurality of facial images. An account management system establishes a facial template for a user based on an image of the user. When the user is approaching a location, the system receives an indication that an identification is desired and receives a plurality of facial images captured by a camera proximate to the location. The system identifies each pupil in a first image of the plurality of facial images and calculates a distance between each pupil. The system compares the calculated distance to a standard distance, the standard distance being a determined distance or distance range between pupils of a person near the location. Based on the comparison, the system determines whether the first image is associated with a user near the location. If not, the method is repeated on one or more other images.

### TECHNICAL FIELD

The present disclosure relates to improving user security and accuracy by using facial image analysis to determine a location of a particular person.

### BACKGROUND

When consumers make conventional purchases at a merchant location, many methods of conducting a transaction are available. Consumers may use many different cards or accounts for purchases, such as gift cards, debit cards, credit cards, stored value cards, loyalty accounts, and other cards or accounts. The user account identifiers and other data represented by the cards may be communicated to the merchant system via magnetic stripes, chips, bar codes, near field communication technologies involving user computing devices, and other suitable mechanisms.

Current applications for conducting transactions at a merchant location may provide an opportunity for the consumer to make a transaction verified via biometric information of a user, such as image recognition of a user at checkout. However, current applications may not adequately prevent inaccurate identification of a user when multiple people are captured in a camera image. When a facial image of a person other than the user is selected, additional steps may be required to accurately complete the transaction. Proper identification of a user is essential to providing secure, accurate, timely and efficient transactions.

### SUMMARY

Techniques herein provide computer-implemented methods to identify a user near a location from among a plurality of facial images. In an example, a system registers with an account management system. The account management system establishes a facial template for a user based on an image provided by the user and establishes a user account. When the user is approaching a location, the system receives an indication that an identification is desired and receives a plurality of facial images captured by a camera proximate to the location. The system identifies each pupil in a first image of the plurality of facial images and calculates a distance between each pupil of the first image. The system compares the calculated distance to a standard distance, the standard distance being a determined distance or distance range between pupils of a person near the location. Based on the comparison, the system determines whether the first image is associated with a user near the location. If not, the method is repeated on one or more other images of the plurality of images.

In certain other example aspects described herein, systems and computer program products to identify users approaching locations from among a plurality of facial images are provided.

These and other aspects, objects, features, and advantages of the examples will become apparent to those having ordinary skill in the art upon consideration of the following detailed description of illustrated examples.

### BRIEF DESCRIPTION OF THE DRAWINGS

FIG. 1 is a block flow diagram depicting a system for processing hands-free transactions with facial recognition of a user, in accordance with certain examples.

FIG. 2 is a block flow diagram depicting a method for processing hands-free transactions with facial recognition of a user, in accordance with certain examples.

FIG. 3 is a block flow diagram depicting a method for registering, by a merchant system, with an account management system and installing hardware at a merchant system location, in accordance with certain examples.

FIG. 4 is a block flow diagram depicting a method for registering, by a user, for an account with an account management system, in accordance with certain examples.

FIG. 5 is a block flow diagram depicting a method for establishing a facial template associated with a user account, in accordance with certain examples.

FIG. 6 is a block flow diagram depicting a method for receiving, by a user computing device, a merchant beacon identifier broadcasted by a merchant beacon device, in accordance with certain examples.

FIG. 7 is a block flow diagram depicting a method for receiving, by a point of sale device, a facial template and a payment token for each user in range of a merchant beacon device, in accordance with certain examples.

FIG. 8 is a block flow diagram depicting a method for initiating, by a user, a transaction at a merchant point of sale device, in accordance with certain examples.

FIG. 9 is a block flow diagram depicting a method for identifying, by a point of sale device, a user via facial recognition, in accordance with certain examples.

FIG. 10 is a block flow diagram depicting a method for identifying, by a point of sale device, which of a plurality of users is attempting to conduct a transaction, in accordance with certain examples.

FIG. 11 is a block flow diagram depicting a method for processing a transaction, a user via facial recognition, in accordance with certain examples.

FIG. 12 is a block flow diagram depicting a computing machine and module, a user via voice recognition, in accordance with certain examples.

### DETAILED DESCRIPTION OF EXAMPLES

### Overview

The examples described herein provide computer-implemented techniques to use facial image analysis to identify when a person is near a location. The identification may be used to conduct a transaction, such as a payment transaction or loyalty program transaction.

In an example, a merchant system registers with an account management system. A merchant may be any entity that facilitates providing goods or services to customers or users. The merchant system installs one or more merchant beacon devices and one or more merchant point of sale devices at a merchant system location. A point of sale device is any device for facilitating interaction with a customer or user. A user establishes an account with the account management system and downloads a user application on a user computing device associated with the user. By way of example, the user application may be a payment application, a loyalty program application or a wallet application. In an example, the user transmits an image of himself and/or an audio recording of himself to the account management system to establish a facial template and/or audio template associated with the user account. The user enters a merchant system location and signs into the user application via the user computing device. The user computing device receives a merchant beacon device identifier broadcasted at the merchant location from the merchant beacon device and transmits the merchant beacon device identifier to the account management system. The account management system may transmit facial templates, audio templates, and/or challenges and responses to the merchant point of sale device associated with users whose user computing devices are in network range of the merchant beacon device and who are signed in to their user applications. The account management system determines user identifiers associated with the users. When a user account is associated with payment functionality, the account management system may generate a payment token for each user whose user computing device is in network range of the merchant beacon device and who is signed in to the payment application. An example payment token comprises a series of alphanumeric and/or symbolic characters. The example payment token may be associated with a payment account of the user and be recognizable by an issuer system associated with the payment account of the user. For example, the account management system generates the payment token and communicates the payment token to an issuer system associated with a payment account of the user along with the user payment account information. In this example, if the issuer system, at a later time, receives the payment token from a point of sale device in a payment transaction, the issuer system is able to extract the user payment account information associated with the payment token. In certain examples, the payment account information is associated with a loyalty account instead of a financial account. The issuer system pays for the product with loyalty points instead of a credit card, debit card, bank account, or other financial account.

A merchant camera device associated with the merchant point of sale device captures a facial image of a user near the merchant point of sale device, and the merchant point of sale device identifies the user based on comparing the captured facial image against the received facial templates. The comparison may occur at any other suitable computing device or system, such as a module on the account management system. In certain instances, the merchant camera may capture more than one face in the image, video, or series of images. For example, if a line is formed at the point of sale device, the image of the person in the front of the line may be captured, but also the person standing second in line may be captured.

The point of sale device, the merchant system, the account management system, or any other suitable system may analyze the image to determine which person in the image is likely the person at the front of the line to conduct a transaction. Throughout the specification, the point of sale device will represent any computing system that analyzes the image. The person that is likely to be attempting to perform a transaction is referred to herein as a transactor.

The point of sale device identifies the pupils of one of the faces in the image. Any other suitable part of the eye may be utilized instead of the pupils. The point of sale device calculates the distance between the pupils in the facial image, such as by counting the number of pixels between the pupils. The point of sale device compares the distance between the pupils in the image to a configured or calibrated standard. The distance of a person from the camera may be estimated based on the distance between the pupils because the distance between the pupils is substantially consistent across a high percentage of the population.

The standard is determined based on the distance between the pupils of a typical user at a determined distance from the camera. The standard may be calculated mathematically, determined based on trial and error, calibrated, or determined in any other suitable manner. The standard may be based on the camera type and/or the image format taken by the camera. If the distance between the pupils in the facial image matches the standard, then the user is determined to be located at the location near the point of sale device. The user is determined to be the user that is attempting the transaction.

After identifying the user, the merchant point of sale device processes a transaction using the identifier associated with the user, such as the payment token, received from the account management system. For payments, the merchant point of sale device generates a transaction authorization request comprising the payment token and transaction details and transmits the transaction authorization request to an issuer system associated with the user account selected for use in the transaction. The issuer system identifies the user payment account based on the received payment token and processes the transaction using the transaction details and the user payment account information. The merchant point of sale device receives an approval of the transaction authorization request and transmits a receipt to the merchant point of sale device.

In certain examples, the transaction conducted based on the user identification is a loyalty account transaction. When the merchant point of sale device or other system identifies the user and the user account, a loyalty transaction is processed using the user account, which may result in a certain number of points, rewards, offers, or any other loyalty information being updated in the user account.

By using and relying on the methods and systems described herein, the account management system, the merchant beacon device, the user computing device, and the merchant point of sale device enable the user to conduct a transaction with the merchant system without the user being required to interact with the user computing device or produce identity documents or physical payment cards, as required in some current technologies. By using facial analysis to determine the position of the user, the methods and systems described herein allow transactions to be conducted securely, accurately and efficiently. As such, the systems and methods described herein may reduce erroneous transactions that must be corrected by chargebacks, additional transaction processing, and unnecessary communications and computer processing.

### Example System Architecture

Turning now to the drawings, in which like numerals indicate like (but not necessarily identical) elements throughout the figures, examples are described in detail.

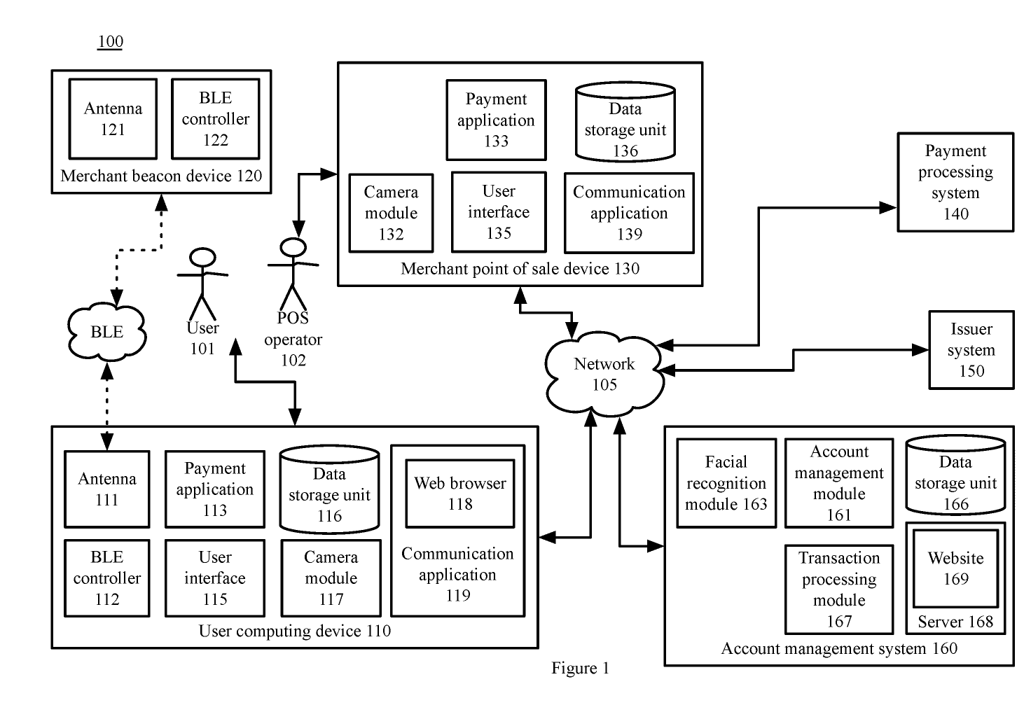

FIG. 1 is a block flow diagram depicting a system 100 for conducting a hands-free transaction with facial recognition of a user 101, in accordance with certain examples. As depicted in FIG. 1, the system 100 includes network computing devices 110, 130, 140, 150, and 160 that are configured to communicate with one another via one or more networks 120. In some embodiments, a user associated with a device must install an application and/or make a feature selection to obtain the benefits of the techniques described herein.

In examples, the network 105 can include a local area network (“LAN”), a wide area network (“WAN”), an intranet, an Internet, storage area network (“SAN”), personal area network (“PAN”), a metropolitan area network (“MAN”), a wireless local area network (“WLAN”), a virtual private network (“VPN”), a cellular or other mobile communication network, Bluetooth, Bluetooth low energy, NFC, or any combination thereof or any other appropriate architecture or system that facilitates the communication of signals, data, and/or messages. Throughout the discussion of examples, it should be understood that the terms “data” and “information” are used interchangeably herein to refer to text, images, audio, video, or any other form of information that can exist in a computer-based environment.

Each network computing device 110, 130, 140, 150, and 160 includes a device having a communication module capable of transmitting and receiving data over the network 105. For example, each network computing device 110, 130, 140, 150, and 160 can include a server, desktop computer, laptop computer, tablet computer, a television with one or more processors embedded therein and/or coupled thereto, smart phone, handheld computer, personal digital assistant (“PDA”), or any other wired or wireless, processor-driven device. In the example depicted in FIG. 1, the network computing devices 110, 130, 140, 150, and 160 are operated by users 101, merchant beacon device 120 operators, merchant point of sale (“POS”) device 130 operators, payment processing system 140 operators, issuer system 150 operators, and account management system 160, respectively.

An example user computing device 110 comprises an antenna 111, a Bluetooth Low Energy (“BLE”) controller 112, a payment application 113, a user interface 115, a data storage unit 116, a camera module 117, a web browser 118, and a communication application 119.

In an example, the antenna 111 is a means of communication between the user computing device 110, a merchant beacon device 120, or other wireless devices. In an example, a BLE controller 112 outputs through the antenna 111 a radio signal, or listens for radio signals from the merchant beacon device 120. In another example a Bluetooth controller, Wi-Fi controller, or a near field communication (“NFC”) controller is used. In an example, the BLE controller 112 outputs through the antenna 111 a radio signal, or listens for radio signals from the merchant beacon device 120.

In an example, the BLE controller 112 is capable of sending and receiving data, performing authentication and ciphering functions, and directing how the user computing device 110 will listen for transmissions from the merchant beacon device 120 or configuring the user computing device 110 into various power-save modes according to BLE-specified procedures. In another example, the user computing device 110 comprises a Bluetooth controller, Wi-Fi controller or an NFC controller capable of performing similar functions. An example BLE controller 112 communicates with the payment application 113 and is capable of sending and receiving data over a wireless, BLE communication channel. In another example, a Bluetooth controller 112, Wi-Fi controller 112, or NFC controller 112 performs similar functions as the BLE controller 112 using Bluetooth, Wi-Fi, or NFC protocols. In an example, the BLE controller 112 activates the antenna 111 to create a wireless communication channel between the user computing device 110 and the merchant beacon device 120. The user computing device 110 communicates with the merchant beacon device 120 via the antenna 111. In an example, when the user computing device 110 has been activated, the BLE controller 112 polls through the antenna 111 a radio signal, or listens for radio signals from the merchant beacon device 120.

In an example, the payment application 113 is a program, function, routine, applet, or similar entity that exists on and performs its operations on the user computing device 110. In certain examples, the user 101 must install the payment application 113 and/or make a feature selection on the user computing device 110 to obtain the benefits of the techniques described herein. In an example, the user 101 may access payment application 113 on the user computing device 110 via the user interface 115. In an example, the payment application 113 may be associated with the account management system 160. In another example, the payment application 113 may be associated with a merchant system associated with the merchant beacon device 120 and/or the merchant point of sale device 130.

In an example, the user interface 115 enables the user 101 to interact with the payment application 113, web browser 118, or any other suitable functions on the user computing device 110. For example, the user interface 115 may be a touch screen, a voice-based interface, or any other interface that allows the user 101 to provide input and receive output from an application or module on the user computing device 110. In an example, the user 101 interacts via the user interface 115 with the payment application 113 and/or web browser 118 to configure user 101 accounts with the account management system 160. In another example, the user 101 interacts via the user interface 115 with the payment application 113 and/or the web browser 118 to enable hands-free payments, if needed.

In an example, the data storage unit 116 comprises a local or remote data storage structure accessible to the user computing device 110 suitable for storing information. In an example, the data storage unit 116 stores encrypted information, such as HTML5 local storage.

In an example, the camera module 117 may be any module or function of the user computing device 110 that captures a digital image. The camera module 117 may be resident on the user computing device 110 or in any manner logically connected to the user computing device 110. For example, the camera module 117 may be connected to the user computing device 110 via the network 105. The camera module 117 may be capable of obtaining individual images or a video scan. Any other suitable image capturing device may be represented by the camera module 117.

In an example, the user 101 can use a communication application 119, such as a web browser 118 application or a stand-alone application, to view, download, upload, or otherwise access documents or web pages via a distributed network 105.

In an example, the web browser 118 can enable the user 101 to interact with web pages using the user computing device 110. In an example, the user 101 may access the user's 101 account maintained by the account management system 160 via the web browser 118. In another example, the user 101 may access a merchant system website or an account management system website 169 via the web browser 118. In certain examples described herein, one or more functions performed by the payment application 113 may also be performed by a web browser 118 application associated with the account management system 160.

In an example, the communication application 119 can interact with web servers or other computing devices connected to the network 105, including a web server of a merchant system and a web server 168 of the account management system 160.

In certain examples, one or more functions herein described as performed by the payment application 113 may also be performed by a web browser 118 application, for example, a web browser 118 application associated with a merchant system website or associated with the account management system 160. In certain examples, one or more functions herein described as performed by the payment application 113 may also be performed by the user computing device 110 operating system. In certain examples, one or more functions herein described as performed via the web browser 118 may also be performed via the payment application 113.

An example merchant beacon device 120 comprises an antenna 121 and a Bluetooth Low Energy (“BLE”) controller 122. In an example, a merchant system location comprises one or more merchant beacon devices 120 installed at the merchant system location. In certain examples, the hardware and functions of the merchant beacon device 120 are encompassed and performed by the merchant POS device 130 or another merchant system device. In certain examples, the merchant beacon device 120 is a stand-alone device that is logically connected or in communication with the merchant POS device 130 or another merchant system device.

In an example, each installed merchant beacon device 120 is associated by an account management system 160 with a particular merchant point of sale device 130 installed at the merchant location. For example, the account management system 160 may comprise a database that correlates merchant beacon device 120 identifiers with merchant POS device 130 identifiers for associated merchant POS devices 130. For example, a merchant POS device 130 identifier may comprise hardware identifier specific to the device such as a serial number or a media access control (“MAC”) identifier. In another example, a merchant beacon device 120 identifier may comprise a hardware identifier specific to the beacon device or an identifier generated by the account management system 160 and stored in the merchant beacon device 120. An example merchant beacon device 120 is programmed to broadcast, emit, or otherwise transmit a particular merchant beacon device 120 identifier over a local wireless network, for example, a BLE network, to any user computing devices 110 within a threshold distance required to maintain the wireless network 105. For example, the wireless network may comprise a BLE network 105, a Wi-Fi network 105, a Bluetooth network 105, an NFC network 105, or any other appropriate wireless network 105.

In an example, the antenna 121 is a means of communication between the user computing device 110 and a merchant beacon device 120. In an example, a BLE controller 122 outputs through the antenna 121 a radio signal, or listens for radio signals from the user computing device 110. In another example a Bluetooth controller, Wi-Fi controller, or a near field communication (“NFC”) controller is used. In an example, the BLE controller 122 outputs through the antenna 121 a radio signal, or listens for radio signals from the user computing device 110.

In an example, the BLE controller 122 is capable of sending and receiving data, performing authentication and ciphering functions, and directing how merchant beacon device 120 will listen for transmissions from the user computing device 110 or configuring the merchant beacon device 120 into various power-save modes according to BLE-specified procedures. In another example, the merchant beacon device 120 comprises a Bluetooth controller, Wi-Fi controller or an NFC controller capable of performing similar functions. An example BLE controller 122 communicates with the payment application 113 and is capable of sending and receiving data over a wireless, BLE communication channel. In another example, a Bluetooth controller 122, a Wi-Fi controller 122, or an NFC controller 122 performs similar functions as the Wi-Fi controller 122 using Bluetooth, Wi-Fi, or NFC protocols. In an example, the BLE controller 122 activates the antenna 121 to create a wireless communication channel between the user computing device 110 and the merchant beacon device 120. The merchant beacon device 120 communicates with the user computing device 110 via the antenna 121. In an example, when the merchant beacon device 120 has been activated, the BLE controller 122 polls through the antenna 121 a radio signal, or listens for radio signals from the user computing device 110.

An example merchant point of sale device 130 comprises a camera module 132, a payment application 133, a user interface 135, a data storage unit 136, and a communication application 139.

In an example, the camera module 132 may be any module or function of the merchant POS device 130 that captures an image or video input of an external environment of the merchant POS device 130. The camera module may be resident on the merchant POS device 130 or in any manner logically connected to the merchant POS device 130. For example, the audio module 131 may be connected to the merchant POS device 130 via the network 105. The camera module 132 may be capable of capturing one or more images or recording a video recording. Any suitable image capturing and/or video recording device may be represented by the camera module 132.

In an example, the payment application 133 is a program, function, routine, applet, or similar entity that exists on and performs its operations on the merchant point of sale device 130. In certain examples, the merchant POS device operator 102 or other merchant system operator must install the payment application 133 and/or make a feature selection on the merchant point of sale device 130 to obtain the benefits of the techniques described herein. In an example, the merchant POS device operator 102 may access the payment application 133 on the merchant POS device 130 via the user interface 135 of the merchant point of sale device 130. In an example, the payment application 133 may be associated with the account management system 160. In another example, the payment application 133 may be associated with a merchant system associated with the merchant beacon device 120 and the merchant camera device 140.

In an example, the user interface 135 enables the merchant POS device operator 102 to interact with the merchant POS device 130. For example, the user interface 135 may be a touch screen, a voice-based interface, or any other interface that allows the merchant POS device operator 102 to provide input and receive output from an application or module on the merchant POS device 130. In an example, the merchant POS device operator 102 interacts via the user interface 135 with the payment application 133.

In an example, the data storage unit 136 comprises a local or remote data storage structure accessible to the merchant POS device 130 suitable for storing information. In an example, the data storage unit 136 stores encrypted information, such as HTML5 local storage.

In an example, the communication application 139, such as a web browser application or a stand-alone application, enables an operator of the merchant POS device 130 to view, download, upload, or otherwise access documents or web pages via a distributed network 105. For example, the communication application 139 may enable communication over the network 105 with the account management system 160, a payment processing system 140, and/or an issuer system 150.

An example payment processing system 140 communicates with the account management system 160 and the merchant point of sale device 130. In an example, when the account management system 160 processes a payment transaction, the account management system 160 transmits user 101 payment account data to the payment processing system 140, which communicates a transaction authorization request an issuer system 150 associated with the payment account data on behalf of the merchant system. In this example, the payment processing system 140 receives an approval or a denial of the payment authorization request from the issuer system 140. In this example, the payment processing system 140 communicates a notification to the account management system 160 and/or the merchant point of sale device 130 of an approved or denied transaction. In this example, the account management system 160 and/or the merchant point of sale device 130 that receives the notification of an approved or denied transaction may transmit receipt data to the user computing device 110. The payment processing system 140 may represent any other card network system that includes an acquirer or other card network system components. The payment processing system 140 may also serve the functions of the issuer system 150 if the payment processing system 140 issued the payment instrument used by the user 101.

An example issuer system 150 approves or denies a payment authorization request received from the merchant point of sale device 130. In an example, the issuer system 150 communicates with the merchant point of sale device 130 over the network 105. In an example, the issuer system 150 communicates with an acquirer system to approve a credit authorization for the user 101 and to make payment to the merchant system. For example, the acquirer system is a third party payment processing system 140. In other examples, the issuer system 150 receives the payment authorization request from the payment processing system 140 or the account management system 160 via the network 105.

An example account management system 160 comprises an account management module 161, a facial recognition module 163, a data storage unit 166, a transaction processing module 167, a server 168, and a website 169.

In an example, the account management module 161 manages one or more user 101 accounts. In an example, a user 101 account may comprise a digital wallet account, an email account, a social networking account, or any other appropriate account associated with the account management system 160. In an example, the account management system 161 communicates with a payment application 113 operating on a user computing device 110 associated with a user 101 having a user 101 account with the account management system 160. In an example, the user 101 enters payment account information into the user account via the payment application 113 and the account management module 161 receives the payment account information over the network 105 and associates the received payment account information with the user account.

In an example, the data storage unit 166 comprises a local or remote data storage structure accessible to the account management system 160 suitable for storing information. In an example, the data storage unit 166 stores encrypted information, such as HTML5 local storage.

In certain examples, the transaction processing module 167 receives transaction details from a merchant POS device 130 and a request to initiate a transaction. Example transaction details comprise merchant system account information, a total amount of the transaction, and a user 101 selection of a user 101 payment account associated with the user's 101 account with the account management system 160. For example, the user's 101 account is a digital wallet account comprising one or more payment account information corresponding to one or more respective payment accounts of the user 101. In an example, the transaction processing module 167 extracts payment account information from the user 101 account corresponding to the user 101 selection of the user 101 payment account received in the transaction details from the merchant POS device 130. In an example, the transaction processing module 167 transmits a payment authorization request to an issuer system 150 or other appropriate financial institution associated with the payment account selected by the user 101 for use in the transaction. An example payment authorization request may comprise merchant system payment account information, user 101 payment account information, and a total amount of the transaction. In an example, after the issuer system 150 processes the payment authorization request, the transaction processing module 167 receives an approval or denial of the payment authorization request from the issuer system 150 over the network 105. In an example, the transaction processing module 167 transmits a receipt to the merchant POS device 130 and/or the user computing device 110 comprising a summary of the transaction.

In certain examples, the functions of the account management system 160 may be performed by the payment processing system 140. For example, the payment processing system 140 may also be the system that manages the payment account and/or the facial recognition functions for the user 101 and/or the merchant system.

It will be appreciated that the network connections shown are example and other means of establishing a communications link between the computers and devices can be used. Moreover, those having ordinary skill in the art having the benefit of the present disclosure will appreciate that the user computing device 110, the merchant beacon device 120, the merchant point of sale device 130, the payment processing system 140, the issuer system 150, and the account management system 160 illustrated in FIG. 1 can have any of several other suitable computer system configurations. For example, a user computing device 110 embodied as a mobile phone or handheld computer may or may not include all the components described above.

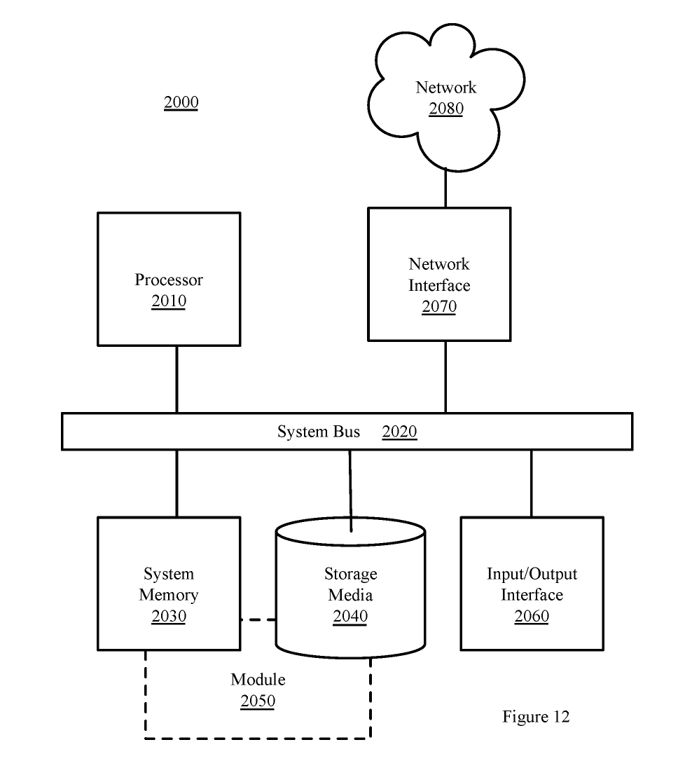

In examples, the network computing devices and any other computing machines associated with the technology presented herein may be any type of computing machine such as, but not limited to, those discussed in more detail with respect to FIG. 12. Furthermore, any functions, applications, or components associated with any of these computing machines, such as those described herein or any other others (for example, scripts, web content, software, firmware, hardware, or modules) associated with the technology presented herein may by any of the components discussed in more detail with respect to FIG. 12. The computing machines discussed herein may communicate with one another, as well as with other computing machines or communication systems over one or more networks, such as network 105. The network 105 may include any type of data or communications network, including any of the network technology discussed with respect to FIG. 12.

### Example Processes

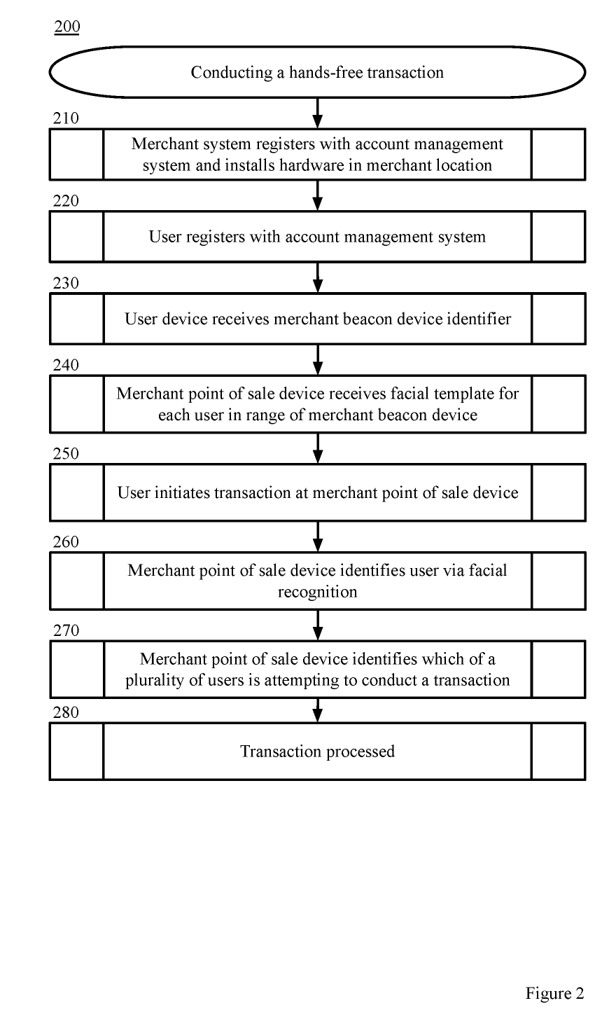

The example methods illustrated in FIGS. 2-11 are described hereinafter with respect to the components of the example operating environment 100. The example methods of FIGS. 2-11 may also be performed with other systems and in other environments.

FIG. 2 is a block flow diagram depicting a method 200 for conducting a hands-free transaction with facial recognition of a user 101, in accordance with certain examples. The method 200 is described with reference to the components illustrated in FIG. 1.

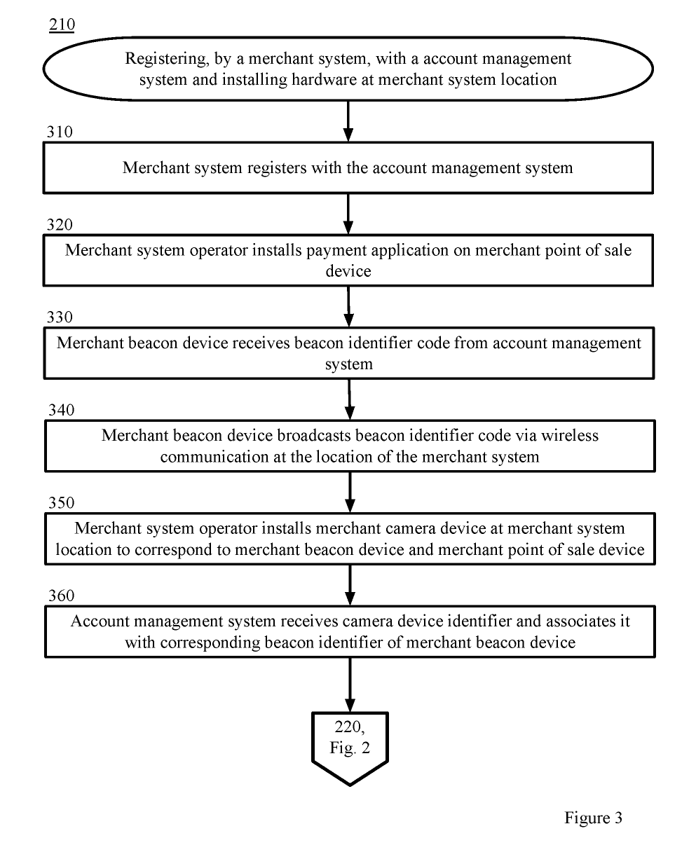

In block 210, the merchant system registers with the account management system 160 and installs hardware in a merchant location. The method for registering, by a merchant system, with an account management system 160 and installing hardware at a merchant system location is described in more detail hereinafter with reference to the method described in FIG. 3.

FIG. 3 is a block flow diagram depicting a method 210 for registering, by a merchant system, with an account management system 160 and installing hardware at a merchant system location, in accordance with certain examples. The method 210 is described with reference to the components illustrated in FIG. 1.

In the examples described herein, the merchant system does not need to install hardware at the example merchant system location in any particular order. The method 210 describes one example method of installing hardware at the merchant location. However, the merchant system or other system installing the merchant hardware does not need to install the merchant POS device 130, the merchant camera device 140, or the merchant beacon device 120 in the order described herein.

In block 310, a merchant system registers with the account management system 160. In an example, an agent of the merchant system accesses an account management system 160 website and registers for a merchant account with the account management system 160 via the website. In an example, the merchant system adds payment account information associated with a merchant account to the merchant account managed by the account management system 160. In an example, the merchant system comprises one or more merchant system locations. For example, the merchant system may comprise one or more physical store locations. An example merchant location comprises one or more merchant point of sale (“POS”) devices 130. In an example, one or more merchant POS device operators 102 operate the one or more merchant POS devices 130 at the merchant system location.

In block 320, a merchant system operator installs the payment application 133 on the merchant point of sale device 130. In another example, the merchant system operator purchases a merchant POS device 130 from the account management system 160 with the payment application 133 pre-installed on the merchant POS device 130. In an example, the merchant POS device 130 is able to communicate with the account management system 160 over a network 105. In an example, the merchant POS device 130 communicates with the account management system 160 via the payment application 133. For example, the merchant POS device 130 may be able to transmit transaction details to the account management system 160 via the payment application 133 over the network 105 to enable the account management system 160 to process a transaction. In another example, the merchant POS device 130 may be able to receive a receipt from the account management system 160 that notifies a merchant POS device operator 102 as to whether a transaction was successful or not.

In block 330, the merchant beacon device 120 receives a beacon identifier from the account management system 160. In an example, the merchant system receives a beacon identifier from the account management system 160 and installs or otherwise saves the beacon identifier on the merchant beacon device 120. In an example, a merchant system operator installs the merchant beacon device 120 in proximity to a merchant POS device 130. In an example, the merchant system operator installs a plurality of merchant beacon devices 120, each merchant beacon device 120 in proximity to one or more associated merchant POS devices 130. In an example, the merchant beacon device 120 is able to broadcast a merchant beacon identifier over a wireless medium, wherein one or more user computing devices 110 located within a threshold proximity to the merchant beacon device 120 are able to receive the merchant beacon identifier over the wireless medium. In another example, the merchant beacon device 120 is able to establish a local network 105 connection to one or more user computing devices 110 located within a threshold proximity to the merchant beacon device 120 and the merchant beacon device 120 transmits the merchant beacon identifier to the one or more user computing devices 110 over the established local network 105 connection. For example, the threshold proximity depends on the network 105 communication protocol utilized by the merchant beacon device 120.

In block 340, the merchant beacon device 120 broadcasts the beacon identifier code via wireless communication at the location of the merchant system. For example, the merchant beacon device 120 may broadcast, emit, or otherwise transmit data comprising the beacon identifier via Wi-Fi, Bluetooth, Bluetooth low energy (“BLE”), near field communication (“NFC”), or other appropriate communication protocol to one or more user computing devices 110 located at the merchant system location within a threshold proximity to the merchant beacon device 120. In some examples, the merchant beacon device 120, at a time before transmitting the merchant beacon identifier, is operable to establish a network 105 connection between the merchant beacon device 120 and one or more user computing devices 110 located at the merchant system location within a threshold proximity to the merchant beacon device 120.

In block 350, a merchant system operator installs the merchant camera device 140 at the merchant system location to correspond to the merchant beacon device 120. In an example, both a merchant camera device 140 and a merchant beacon device 120 are installed in proximity to a particular merchant POS device 130. In another example, a merchant camera device 140 and a merchant beacon device 120 are installed in proximity to two or more particular merchant POS devices 130. In another example, the merchant beacon device 120 is positioned at the entrance to the merchant location or in a centrally located position in the merchant location. In this position, the user computing device 110 is able to prepare for a transaction at a time before the user 101 approaches the POS device 130.

In an example, the merchant camera device 140 is oriented to be able to capture video and/or images of a face of a user 101 standing in front of one or more merchant POS devices 130 during the process of checkout. In an example, the merchant system installs a merchant camera device 140 that is oriented to capture video and/or images of the face of a user standing in front of a particular merchant POS device 130. In another example, the merchant system installs a merchant camera device 140 that is oriented to capture video and/or images of the faces of one or more users 101 standing within a proximity of a particular plurality of merchant POS devices 130 within a range of a field of vision of the camera module 147 of the merchant camera device 140.

In another example, multiple camera devices 140 are installed at the location of the merchant. For example, one camera device 140 may be located at the entrance to capture users 101 as they enter the store, and then a second camera device 140 located at the POS device 130 to capture users 101 as they approach the POS device 130 to conduct a transaction.

In block 360, the account management system 160 receives a merchant camera device 140 identifier and associates it with the corresponding beacon identifier code of the merchant beacon device 120. In an example, the merchant system and/or the account management system 160 configures the merchant camera device 140 so that the merchant camera device 140 is able to communicate with the account management system 160 over the network 105. An example camera device 140 identifier comprises a hardware identifier, a MAC address, or other useful or relevant identifier associated with the merchant camera device 140. In an example, the account management system 160 comprises a database comprising merchant camera device 140 identifiers and associated beacon identifiers for merchant beacon device 120 identifiers for a particular merchant system location. In an example, the merchant camera device transmits the merchant beacon device 120 identifier in addition to the merchant camera device 140 identifier to the account management system 160. In an example, the merchant camera device 140, during the setup and installation process, may receive the merchant beacon device 120 identifier over an appropriate wireless communication channel from the merchant beacon device 120. In another example, the merchant camera device 140, during the setup and installation process, may establish a network 105 connection with the merchant beacon device 120 and receive the merchant beacon device 120 identifier over the network 105. In another example, the account management system 160 receives the merchant camera device 140 identifier, extracts one or more merchant beacon device 120 identifiers from the database, and associates the merchant camera device 140 identifier with one or more of the one or more extracted merchant beacon device 120 identifiers. In yet another example, the merchant system operator installs the one or more merchant beacon devices 120 after installing the one or more merchant camera devices 140. In this example, the account management system 160 generates a merchant beacon device identifier to associate with a merchant camera device 140 identifier and transmits the generated merchant beacon device identifier to the merchant system. In this example, the merchant system operator manually configures the merchant beacon device 120 to broadcast, emit, or otherwise transmit the merchant beacon device identifier assigned by the account management system 160 over a network 105.

In certain examples, one or both of the merchant camera device 140 and the merchant beacon device 120 are components of the merchant POS device 130 or are wirelessly or physically connected to the merchant POS device 130 and controlled by one or more processors of the merchant POS device 130. In certain examples, certain functions described herein as performed by the merchant camera device 140 and/or the merchant beacon device 120 may also be performed by the merchant POS device 130.

From block 360, the method 210 proceeds to block 220 of FIG. 2.

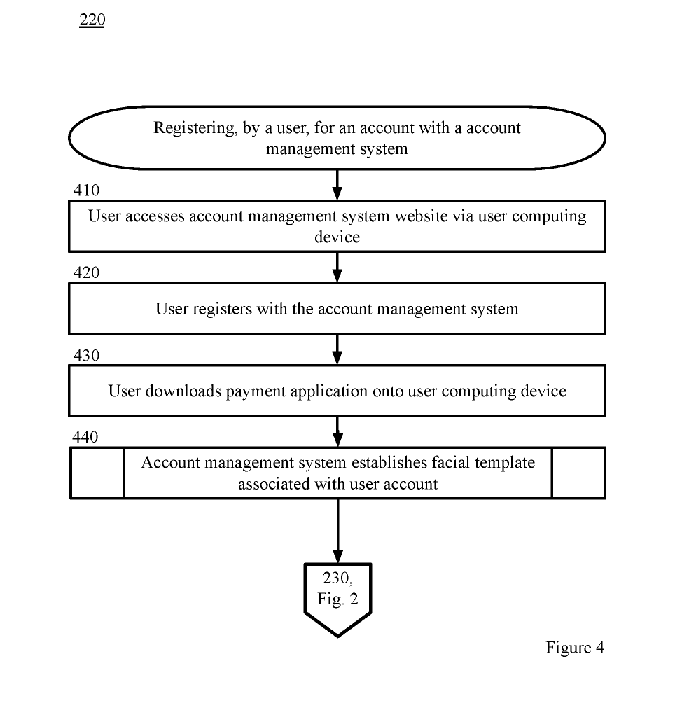

Returning to FIG. 2, in block 220, the user 101 registers with the account management system 160. The method for registering, by a user 101, for an account with an account management system 160 is described in more detail hereinafter with reference to the method 220 described in FIG. 4.

FIG. 4 is a block flow diagram depicting a method 220 for registering, by a user 101, for an account with an account management system 160, in accordance with certain examples. The method 220 is described with reference to the components illustrated in FIG. 1.

In block 410, the user 101 accesses the account management system website 169. For example, the user 101 accesses the account management system 160 via the web browser 118 of the user computing device 110. In another example, the user 101 may otherwise contact the account management system 160 to register for a user 101 account.

In block 420, the user 101 registers with the account management system 160. The user 101 may obtain a user account number, receive the appropriate applications and software to install on the user computing device 110, request authorization to participate in hands-free payment processing, or perform any action required by the account management system 160. The user 101 may utilize the functions of the user computing device 110, such as the user interface 115 and the web browser 118, to register and configure a user 101 account. In an example, the user 101 may enter payment account information associated with one or more user 101 accounts, for example, one or more credit accounts, one or more bank accounts, one or more stored value accounts, and/or other appropriate accounts into the user 101 account maintained by the account management system 160.

In block 430, the user 101 downloads the payment application 113 onto the user computing device 110. In an example, the payment application 113 operating on the user computing device 110 is able to communicate with the account management system 160 over the network 105. In an example, the user 101 may configure user 101 account settings or add, delete, or edit payment account information via the payment application 113. In an example, the user 101 may select an option to enable or disable the permission of the account management system 160 to process hands free transactions. For example, a hands free transaction comprises a transaction wherein the user 101 does not need to interact with the user computing device 110 or requires minimal user 101 interaction with the user computing device 110 to initiate a transaction with the merchant system.

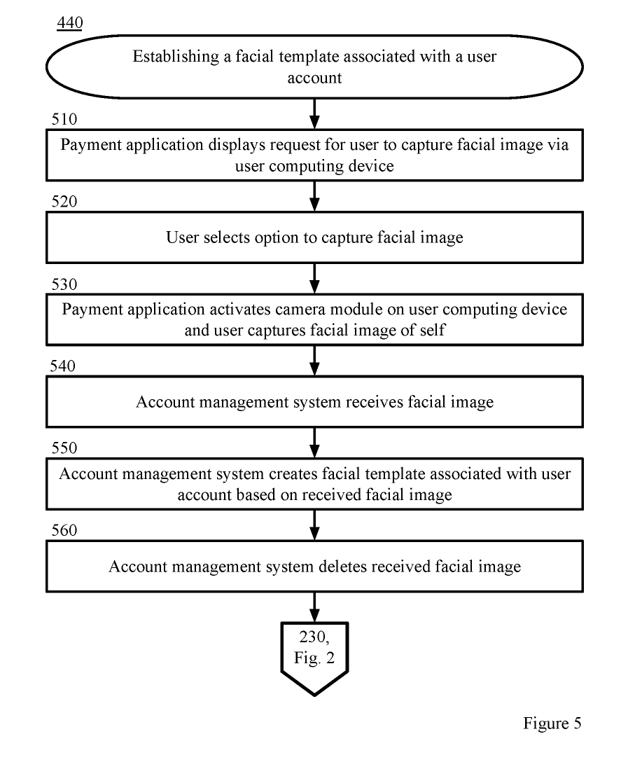

In block 440, the account management system 160 establishes a facial template associated with the user 101 account. The method for establishing a facial template associated with a user 101 account is described in more detail hereinafter with reference to the method 440 described in FIG. 5.

FIG. 5 is a block flow diagram depicting a method 440 for establishing a facial template associated with a user 101 account, in accordance with certain examples. The method 440 is described with reference to the components illustrated in FIG. 1.

In block 510, the payment application 113 displays a request for the user 101 to capture a facial image via the user computing device 110. In an example, the payment application 113 displays the request via the user interface 115. In an example, the user interface 115 may display a request that reads, “to enable hands free transactions, we need an image of your face. Would you like submit a facial image now?” In this example, the user 101 may select an option to take a current picture or may otherwise select a picture stored on the user computing device 110.

In block 520, the user 101 selects an option to capture a facial image. For example, the user 101 actuates an object on the user interface 115 that reads, “yes, I would like to take a picture now.”

In block 530, the payment application 113 activates the camera module 117 on the user computing device 110 and the user 101 captures a facial image. In an example, the user computing device user interface 115 may display a live camera feed of the user 101 to aid the user 101 in aligning the user's 101 face to take the facial image. In an example, the payment application 113 may display on the user computing device 110 a box or other perimeter on the user interface 115 within which the user 101 should align his face to take a picture of a required size predetermined by the account management system 160. In an example, the user 101 may actuate an object on the user interface 115 to capture the image. In this example, in response to the user actuating the object on the user interface 115, the camera module 117 receives a command from the payment application 113 to capture an image of the user 101. In another example, the camera module 117 receives a command from the payment application 113 to capture a plurality of images of the user 101 as the user 101 moves the camera around the user's 101 face. For example, each of the plurality of images of the user 101 may correspond to a particular pose of the user's 101 face. An example facial image may comprise a digital image of the face of a user 101. In an example, the account management system 160 may establish guidelines for users 101 in submitting facial images. For example, the payment application 113 may direct the user 101 to remove any hats, head coverings, glasses, or other objects or accessories that may occlude regions of the user's 101 face so that payment application 160 may receive a complete depiction of the user's 101 face.

In an example, the user computing device 110 determines if the captured facial image is a valid facial image or an invalid facial image. For example, a valid facial image complies with guidelines predetermined by the account management system 160 and an invalid facial image does not comply with one or more of the guidelines. For example, if the user computing device 110 captures a facial image that comprises incorrect dimensions, if part or all of the user's 101 face is occluded, or if the image is too dark or too bright, the user computing device 110 rejects the invalid facial image and displays a request directing the user 101 to capture a subsequent facial image. In this example, the user 101 captures a subsequent facial image via the user computing device 110, and the user computing device 110 transmits the subsequent facial image to the account management system 160 via the network 105.

In block 540, the account management system 160 receives the facial image. In another example, the account management system 160 receives a plurality of facial images of the user 101. For example, the payment application 113 transmits the one or more facial images of the user 101 to the account management system 160 via the network 105. In an example, the account management system 160 associates the received one or more facial images with the user 101 account. For example, the account management system 160 is able to identify the user 101 account to associate with the received one or more images because the user 101 is currently logged in to the payment application 113 on the user computing device 110 at the time the one or more facial images are transmitted to the account management system 160. In certain examples, the account management system 160 determines if the received facial image is a valid facial image or an invalid facial image. For example, a valid facial image complies with all guidelines predetermined by the account management system 160 and an invalid facial image does not comply with one or more of the guidelines. For example, if a user 101 submits a facial image that comprises incorrect dimensions, if part or all of the user's 101 face is occluded, or if the image is too dark or too bright, the account management system 160 rejects the invalid facial image and transmits a request to the user computing device 110 directing the user 101 to capture a subsequent facial image to transmit to the account management system 160. In this example, the user computing device 110 receives and displays the request, the user 101 captures a subsequent facial image via the user computing device 110, and the user computing device 110 transmits the subsequent facial image to the account management system 160 via the network 105.

In another example, the user 101 submits a facial image that is not of a face and the account management system 160 or payment application 113 determines, via facial recognition, that the image is not of a face, the account management system 160 or payment application 113 rejects the invalid facial image and transmits a request to the user computing device 110 for display by the user computing device 110 directing the user 101 to capture a subsequent facial image to transmit to the account management system 160. In this example, the user computing device 110 receives and displays the request, the user 101 captures a subsequent facial image via the user computing device 110, and the user computing device 110 transmits the subsequent facial image to the account management system 160 via the network 105.

In yet another example, the user 101 submits a facial image, but the account management system 160 or payment application 113 determines that the image, based on one or more image metrics such as image resolution, is not of a minimum quality standard, and the account management system 160 or payment application 113 rejects the invalid facial image and transmits a request to the user computing device 110 for display by the user computing device 110 directing the user 101 capture a subsequent facial image to transmit to the account management system 160. In this example, the user computing device 110 receives and displays the request, the user 101 captures a subsequent facial image via the user computing device 110, and the user computing device 110 transmits the subsequent facial image to the account management system 160 via the network 105.

In block 550, the account management system 160 creates a facial template associated with the user 101 account based on the received facial image. In another example, the account management system 160 generates a corresponding facial template for each of a plurality of received facial images associated with the user 101 account. In an example, the facial template is of a predetermined size, for example, a 128-byte facial template. In an example, the account management system 160 generates a facial template comprising a computer code representation of the digital facial image. For example, the facial template may describe key features of the facial image of the user 101, such as shape, color, line, value, space, form, texture, or other useful or relevant feature of the image or of particular regions of the image. In an example, the facial template is generated by processing the facial image through a convolutional neural network. In an example, the account management system 160 stores the generated facial template associated with the user 101 in a data storage unit 166 associated with the account management system 160. For example, the account management system 160 database may comprise a table or other means by which it correlates each user 101 account identifier with an associated facial template of the user 101.

In another example, after the user computing device 110 captures one or more facial images of the user 101, the user computing device 110 generates one or more facial templates corresponding to one or more of the one or more captured facial images of the user 101. In this example, the user computing device 110 transmits the one or more generated facial templates to the account management system 160 over the network 105.

In block 560, the account management system 160 deletes the received facial image. For example, the account management system 160 only uses a facial template comprising a computer code representation of the facial image of the user 101. In another example, the account management system 160 saves the received facial image for future processing. For example, the account management system 160, at a later time, updates a facial template generation algorithm and generates an updated facial template corresponding to the saved facial image.

From block 560, the method 440 proceeds to block 230 in FIG. 2.

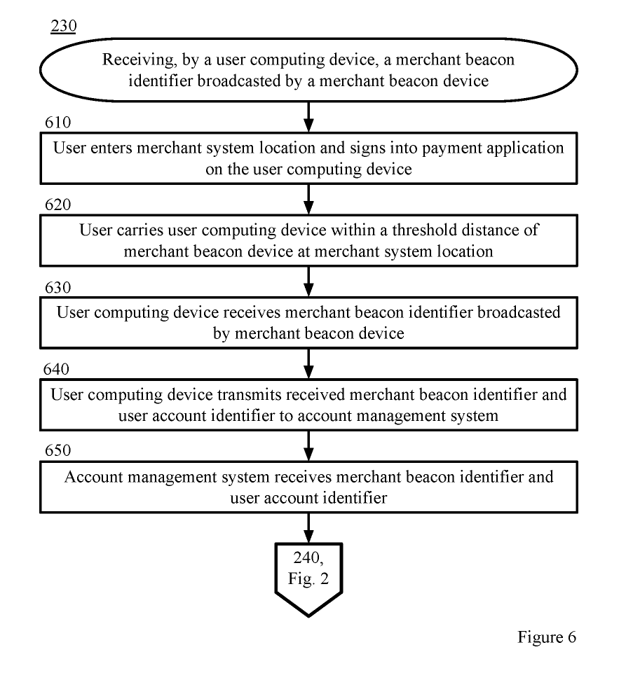

Returning to block 230, in FIG. 2, the user computing device 110 receives a merchant beacon device 120 identifier. The method for receiving, by a user computing device 110, a merchant beacon identifier broadcast by a merchant beacon device 120 is described in more detail hereinafter with reference to the method 230 described in FIG. 6.

FIG. 6 is a block flow diagram depicting a method 230 for receiving, by a user computing device 110, a merchant beacon identifier broadcast by a merchant beacon device 120, in accordance with certain examples. The method 230 is described with reference to the components illustrated in FIG. 1.

In block 610, the user 101 enters the merchant system location and signs into the payment application 113 on the user computing device 110. In another example, the user 101 signs into the payment application 113 at a time before entering the merchant system location and enters the merchant location carrying the user computing device 110 signed into the payment application 113. In another example, the payment application 113 is automatically signed into based on other authentication technologies. The payment application 113 may be activated manually by the user 101 or automatically when the user computing device 110 recognizes the beacon identifier.

In an example, the user 101 may have a username and password associated with the user 101 account maintained by the account management system 160. In an example, the user 101 opens the payment application 113 on the user computing device 110 and enters a username and/or password via the user interface 115 to sign in to the payment application 113. In an example, when the user 101 is signed in to the payment application 113, the payment application is able to communicate with the account management system 160 over the network 105. In this example, when the user 101 is not signed in to the payment application 113, the payment application does not communicate with the account management system 160 even if the a network 105 connection is available. In an example, the user 101 may sign out of the payment application 113 at any time by actuating one or more objects on the user interface 115 of the user computing device 110. In an example, after signing in to the payment application 113, the user 101 configure one or more user 101 account settings, add, edit, or delete user 101 payment account information, and/or change user 101 preferences. In certain examples, a user 101 may be required to make a feature selection to obtain the benefits of the techniques described herein. For example, the user 101 may have to enable one or more user 101 account settings to enable hands free transactions according to the methods described herein.

In an example, payment application 113 may provide options, data, configurable alerts, and other suitable features to the user 101. For example, the payment application 113 may comprise a listing of merchant systems and merchant locations that participate in hands free payment transactions according to one or more of the methods described herein. The listing may be updated periodically from the account management system 160. The payment application 113 may notify the user 101 when the user 101 is within a configured vicinity of a participating merchant system. The payment application 113 may provide the user 101 with options to update payment preferences. The payment application 113 may provide the user 101 with a listing of recent transactions. The payment application 113 may provide any other suitable information to the user 101.

In block 620, the user 101 carries the user computing device 110 within a threshold distance of the merchant beacon device 120 at the merchant system location. In an example, the user 101 enters a location of the merchant system. The user 101 may enter the merchant location carrying the user computing device 110 in a pocket or a bag, in the hands of the user 101, or in any suitable manner. The location of the merchant system may be a store location, a kiosk location, or any suitable physical location of a merchant system. In another example, a merchant POS operator 102 may be mobile and arrive at a location of the user 101. For example, the merchant system may be a restaurant and the merchant POS device operator 102 may be a delivery person possessing a portable merchant POS device 130.

In certain examples, the payment application 113 may alert the user 101 when the user 101 is in the vicinity of a merchant system that accepts hands-free payments. The alert may be provided via a message on the user computing device 110, via an email or a text, or in any suitable manner. In an example, the alert may be based on the location of the user 101 as determined by a GPS module (not depicted) resident on the user computing device 110. For example, the payment application 113 accesses the GPS data from the GPS module and compare the GPS location to a list of locations of merchant systems that accept hands free payments. For example, the payment application 113 comprises a list or accesses a list maintained by the account management system 160 of merchant system locations that accept hands free payments. If a match results from the comparison, then an alert is generated and provided to the user 101. The match may result if the user 101 is within a configured distance of a qualified merchant system location. In an example, the alerts may be configured to alert in any suitable manner. In an example, the alerts may be combined in commercially dense environments or the alerts may be presented individually. In another example, the alerts may be configured to only alert the user 101 a configured number of times. For example, the alert may be presented three times, but upon a fourth instance, the alert is not presented. The alerts may be presented as a notification with an audible alert, a vibration, a popup alert on the user interface 115 of the user computing device 110, or other suitable alert.

In block 630, the user computing device 110 receives a merchant beacon identifier broadcast by the merchant beacon device 120. The user computing device 110 recognizes a merchant beacon device 120 via wireless communication at the location of the merchant system. The user computing device 110 may be configured to search for beacons or other wireless signals. In an example, the user computing device 110 and the merchant beacon device 120 establish a BLE wireless network 105 connection. In other examples, the user computing device 110 and the merchant beacon device 120 establish a Bluetooth, Wi-Fi, NFC, or other appropriate network 105 connection. Upon entering the range of the signal of the merchant beacon device 120, the user computing device 110 receives the merchant beacon identifier.

In block 640, the user computing device 110 transmits the received merchant beacon identifier and a user 101 account identifier to the account management system 160. In an example, the user computing device 110 transmits the data received in the merchant beacon identifier along with a user 101 account identifier to the account management system 160 over the network 105.

In block 650, the account management system 160 receives the merchant beacon identifier and the user 101 account identifier. For example, the account management system 160 receives the merchant beacon identifier and the user 101 account identifier over the network 105. The user computing device 110 may compare the data from the merchant beacon identifier to a database of merchant beacon identifier data and merchant camera device identifier data to determine an identity of the merchant system and merchant camera device 140 associated with the merchant beacon identifier and/or to verify the authenticity of the beacon.

From block 650, the method 230 proceeds to block 240 in FIG. 2.

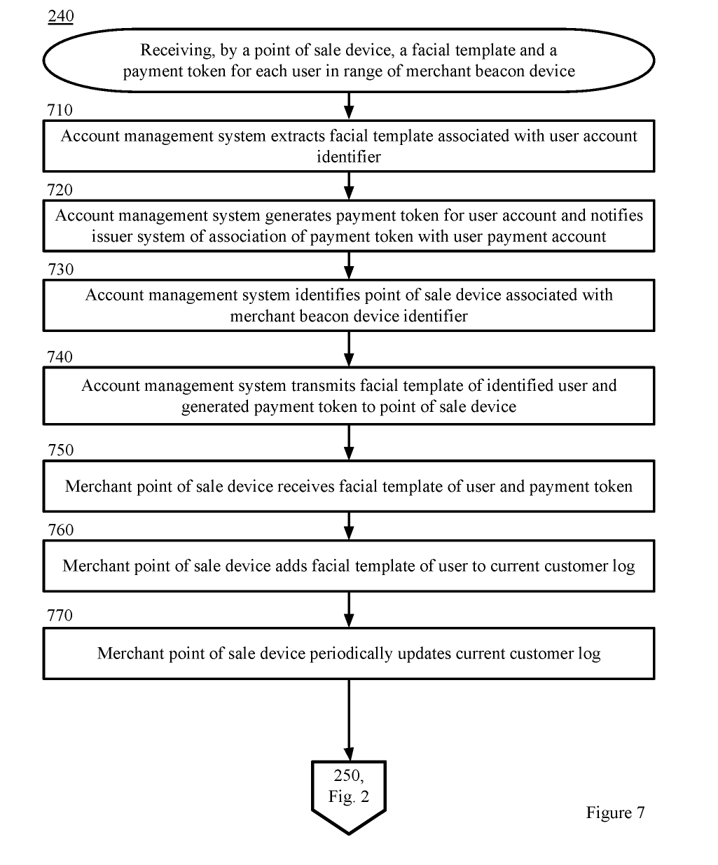

Returning to FIG. 2, in block 240, the merchant point of sale device 130 receives a facial template for each user 101 in range of the merchant beacon device 120. The method for receiving, by a merchant camera device 140, a facial template for each user 101 in range of the merchant beacon device 120 is described in more detail hereinafter with reference to the method 240 described in FIG. 7.

FIG. 7 is a block flow diagram depicting a method 240 for receiving, by a merchant camera device 140, a facial template for each user 101 in range of the merchant beacon device 120, in accordance with certain examples. The method 240 is described with reference to the components illustrated in FIG. 1.

In block 710, the account management system 160 extracts a facial template associated with the user account identifier. In an example, the account management system 160 accesses a database comprising stored facial templates of a plurality of users 101 with corresponding user account identifiers for each user 101. For example, this database is stored in the data storage unit 166. The account management system 160 identifies the facial template associated with the user account identifier and prepares the identified facial template for communication or use.

In block 720, the account management system 160 generates a payment token for a user payment account and notifies an issuer system of association of the payment token with the user payment account. In an example, the account management system 160 generates a payment token for each user 101 whose user computing device 110 is in network range of the merchant beacon device 120 and who is signed in to the payment application 113. An example payment token comprises a series of alphanumeric and/or symbolic characters. The example payment token may be associated with a payment account of the user 101 and be recognizable by an issuer system 150 associated with the payment account of the user 101. For example, the account management system 160 generates the payment token and communicates the payment token to an issuer system 150 associated with a payment account of the user 101 along with the user 101 payment account information. In this example, if the issuer system 150, at a later time after receiving the payment token from the account management system 160, receives the payment token from a point of sale device 130 in a payment transaction, the issuer system 150 is able to extract the user 101 payment account information associated with the payment token.

In some examples, the account management system 160 may place restrictions on payment tokens for security reasons or according to one or more configurations of the user 101 account desired by the user 101. For example, the payment token may only be valid for a preconfigured length of time, for example, one hour. In another example, the payment token may only be valid for use in a transaction between the user 101 and a particular merchant system. In yet another example, the payment token is only valid for use within a particular geographic boundary or within a threshold distance from a geographic point. In an example, the account management system 160 communicates one or more of these example restrictions to the issuer system 150 along with the payment token and the issuer system 150 associates these one or more restrictions with the payment token and the user 101 payment account data in a database of the issuer system 150.

In an example, the account management system 160 may communicate, to the issuer system 150 along with the payment token and the user 101 account data, a current time stamp representing a time when the payment token was generated to associate with the payment token. In another example, the account management system 160 may communicate, to the issuer system 150 along with the payment token and the user 101 account data, location data describing geographic boundaries and/or threshold distances from geographic points where the payment token may be used in a transaction.

In yet another example, the account management system 160 may communicate, to the issuer system 150 along with the payment token and the user 101 account data, a merchant system identifier and instructions that only payment authorization requests originating from merchant systems comprising the merchant system identifier may be approved. In an example, the issuer system 150 associates the payment token, the user 101 payment account data associated with the payment token, the one or more restrictions placed on the payment token by the account management system 160, and/or one or more of location data, time stamp data, merchant system identifier data, or other data that the issuer system 150 may use to determine whether the one or more restrictions on the payment token are satisfied to enable use of the payment token.

In another example, the payment token is generated by the payment application 113 on the user computing device 110 and communicated to the merchant POS device 130 or to the account management system 160. The generation of the token may follow similar processes and rules as described herein with tokens generated by the account management system 160.

In another example, the payment token is associated with a loyalty account of the user 101. The user 101 in this example, may purchase items using loyalty points or offers. The loyalty purchases may be in conjunction with payment account transactions or exist as a separate system.

In block 730, the account management system 160 identifies a merchant point of sale device 130 associated with the merchant beacon device 120 identifier. In an example, the account management system 160 recognizes that the merchant beacon identifier is associated with the account management system 160 and a particular merchant point of sale device 130 at the merchant system location. In an example, the account management system 160 recognizes that the merchant beacon identifier is associated with a plurality of merchant point of sale device 130 installed at a particular merchant location.

In block 740, the account management system 160 transmits the identified facial template of the identified user 101 along with the generated payment token to the merchant point of sale device 130 associated with the merchant beacon device 120 identifier. In another example, the account management system 160 transmits the facial template of the identified user 101 and the generated payment token to a plurality of merchant point of sale devices 130 associated with the merchant beacon device 120 identifier. In certain examples, the account management system 160 receives, in real time, a plurality of transmissions from user computing devices 101 corresponding to a plurality of users 101 present at the merchant system location, each transmission comprising a user 101 account identifier and a retransmitted merchant beacon identifier. In these examples, the account management system 160 retrieves, in response to receiving each such transmission, a facial template associated with the received user 101 account identifier and transmits a facial template to one or more merchant point of sale devices 130 at the merchant location associated with the merchant beacon identifier.

In block 750, the merchant point of sale device 130 receives the facial template of the user 101. In another example, in addition to or instead receiving the facial template, the merchant point of sale device 130 receives an audio template and/or a challenge and response associated with the user 101 account. In another example, a plurality of merchant point of sale devices 130 receive the facial template of the user 101. In yet another example, the merchant point of sale devices and/or the plurality of merchant point of sale devices 130 receives additional facial templates from the account management system 160 corresponding to one or more users other than the instant user 101 having user computing devices 110 in network 105 connection to a merchant beacon device 120 according to the method previously described herein. For example, the one or more additional facial templates are received in real time from the account management system 160 as additional users 101 other than the instant user 101 receive the merchant beacon device 120 identifier over a wireless communication network 105 or otherwise establish a network 105 connection between their user computing devices 110 and one or more merchant beacon devices 120. For example, the one or more merchant point of sale devices 130 may receive one or more additional facial templates corresponding to one or more additional users 101 at a time before, at the same time, or after the time at which the merchant point of sale devices 130 receives the facial template of the instant user 101.

In block 760, the merchant point of sale device 130 adds the facial template of the user 101 to a current customer log. In an example, the current customer log is accessible by the merchant point of sale device 130 and by the account management system 160. In an example, the merchant point of sale device 130 maintains the current customer log on the merchant point of sale device 130 or on a computing device logically connected to the merchant point of sale device 130.

In block 770, the merchant point of sale device 130 periodically updates the current customer log. The merchant point of sale device 130 is notified by the account management system 160 as users 101 signed into a payment account enter or leave a network range of the merchant beacon device 120. From block 770, the method 240 returns to block 250 of FIG. 2.

Returning to block 250, in FIG. 2, the user 101 initiates a transaction at the merchant POS device 130.

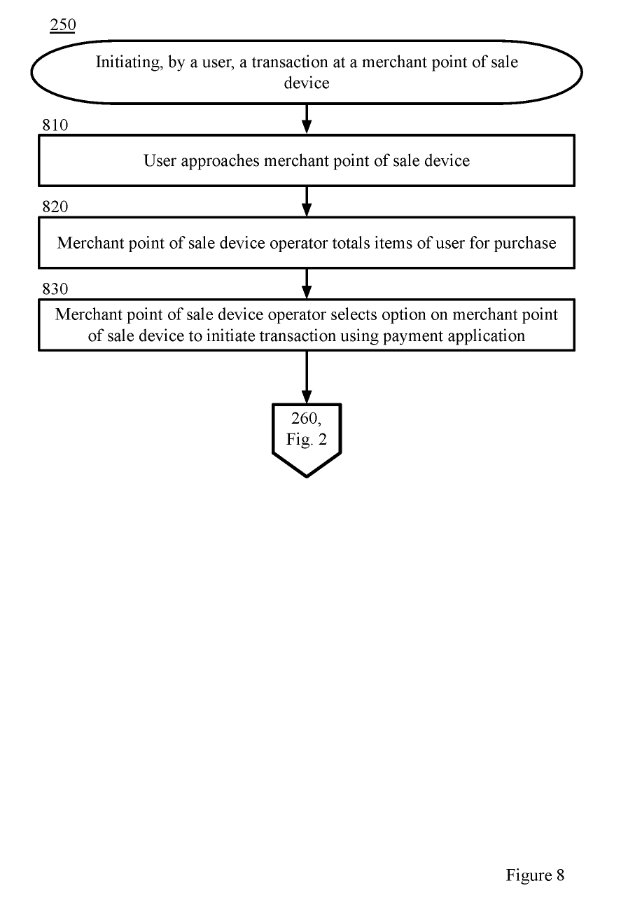

The method for initiating, by a user 101, a transaction at a merchant point of sale device 130 is described in more detail hereinafter with reference to the method 250 described in FIG. 8. In the examples described herein, the user 101 initiates a “hands free transaction” at the merchant POS device 130. An example hands free transaction does not require any interaction with the user computing device 110 on the part of the user 101. In another example, a hands free transaction requires only minimal interaction with the user computing device 110 by the user 101.

FIG. 8 is a block flow diagram depicting a method 250 for initiating, by a user 101, a transaction at a merchant POS device 130, in accordance with certain examples. The method 250 is described with reference to the components illustrated in FIG. 1.

In block 810, the user 101 approaches the merchant point of sale device 130. In an example, at a time prior to approaching the merchant POS device 130, the user 101 browses the merchant system location and selects one or more items to purchase. In this example, the user 101 may collect the one or more items and carry, or otherwise transport the one or more items to the merchant POS device 130. Throughout the example, the items purchased may be tangible or non-tangible items, such as services.

In block 820, the merchant point of sale device 130 operator 102 totals the items of the user 101 for purchase. In an example, the merchant POS device operator 102 scans barcodes attached to the one or more items or otherwise enters descriptions and prices associated with the one or more items into the merchant POS device 130. In an example, after scanning or manually entering the items into the merchant POS device 130, the merchant POS device operator 102 actuates an object on the user interface 135 of the merchant POS device 130 to order the merchant POS device 130 to total the items. In an example, the merchant POS device 130 displays, via the user interface 135, the total to the user 101.

In block 830, the merchant point of sale device 130 operator asks the user 101 to select a payment option. In an example, the merchant POS device 130 displays one or more payment options that the user 101 may select to use in a transaction. Example payment options may comprise payment via a payment application 113 associated with the account management system 160, payment by cash, payment by check, payment by credit card, payment by debit card, and/or any other means of payment that the merchant system can or is willing to accept for payment from the user 101. In an example, the one or more payment options are displayed as objects on the user interface 135 and are selectable by the merchant POS device operator 102 in response to the user 101 directing the merchant POS device 102 operator to make a selection.

In block 840, the user 101 directs the merchant point of sale device operator 102 to initiate a hands-free transaction via the payment application 113. In an example, in response to receiving a verbal request from the user 101 to select the payment application 113 as a payment option, the merchant POS device operator 102 actuates an object on the user interface 135 of the merchant POS device 130 corresponding to the payment application 113 payment option. In certain examples, the hands-free transaction is the only option available, and a direction from the user 101 to the operator 102 is not necessary.

In block 850, the merchant point of sale device operator 102 selects an option on the merchant point of sale device 130 to initiate a transaction using the payment application 113. In an example, the merchant POS device 130 displays a confirmation screen after the merchant POS device operator 102 selects an option to initiate a transaction using the payment application 113. An example confirmation screen may display information summarizing the potential transaction and comprising one or more of a transaction total, a description of the one or more items being purchased by the user 101, and a indication that the user 101 selected the payment application 113 as the method of payment for the transaction. An example confirmation screen may further display options to confirm the transaction or cancel the transaction. In an example, the user 101 reviews the confirmation screen, determines that the information displayed on the confirmation screen is correct, determines to continue with the transaction, and directs the merchant POS device operator 102 to select the option to confirm the transaction via the user interface 135.

From block 850, the method 250 proceeds to block 260 in FIG. 2.

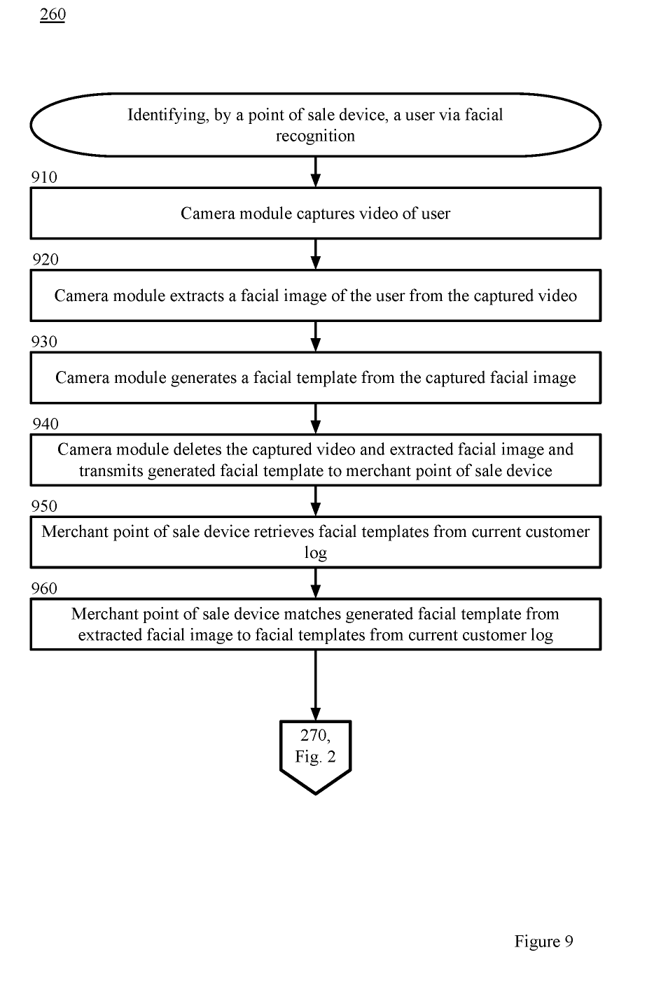

Returning to FIG. 2, in block 260, the merchant point of sale device 130 identifies the user 101 via facial recognition. The method for identifying, by a merchant point of sale device 130, a user 101 via facial recognition is described in more detail hereinafter with reference to the method 260 described in FIG. 9. In other examples, the merchant point of sale device 130 identifies the user 101 via audio recognition and/or via a challenge and response.

FIG. 9 is a block flow diagram depicting a method 260 for identifying, by a merchant point of sale device 130, a user 101 via facial recognition, in accordance with certain examples. The method 260 is described with reference to the components illustrated in FIG. 1.

In block 910, a camera module 132 of the merchant point of sale device 130 captures video of the user 101. In an example, in response to receiving a request to identify the user 101, the merchant point of sale device 130 activates the camera module 132 to begin to capture a video of the surroundings of the merchant point of sale device 130. In an example, the merchant POS device 130 captures a video feed of the user's 101 face. In another example, the camera module 132 continuously captures, but does not record, a video feed of its surroundings. In this example, when the merchant point of sale device 130 receives an input from the merchant POS device 130 operator 102, a request to identify the user 101 from the account management system 160, the camera module 132 beings to record the video feed for a threshold amount of time. In an example, the user 101 may be moving during the period in which the camera module 132 records the video feed. In an example, the camera module 132 extracts a facial image by determining a particular frame of the video feed and area of the instance of the video feed corresponding to the face of the user.

In block 920, the camera module 132 extracts a facial image of the user 101 from the captured video. In an example, the camera module 132 determines a frame of the captured video to provide an image of the user's 101 face and extracts the frame of the captured video comprising the facial image of the user 101.

In certain other examples, the camera module 132 identifies a frame of the captured video to provide an image of the faces of a plurality of users 101. For example, the frame comprises an image of the face of a first user 101, a second user, and a third user at different locations in the image. In this example, one camera module 132 associated with a particular merchant point of sale device 130 may capture video of an environment corresponding to an area in the proximity of multiple merchant POS devices 130. In this example, the camera module 132 may determine to which particular merchant POS device 130 each of the plurality of faces of the corresponding plurality of users 101 in the extracted image.

In block 930, the camera module 132 generates a facial template from the captured facial image. In another example, the merchant point of sale device 130 generates the facial template. In an example, the facial template is of a predetermined size, for example, a 128-byte facial template. In an example, the account management system 160 generates a facial template comprising a computer code representation of the digital facial image. For example, the facial template may describe key features of the facial image of the user 101, such as shape, color, line, value, space, form, texture, or other useful or relevant feature of the image or of particular regions of the image. In another example, the facial template is generated by processing the facial image through a convolutional neural network. In an example, the camera module 132 stores the generated facial template in a data storage unit 146 associated with the merchant point of sale device 130. For example, the camera module 132 database may comprise a log of facial templates of current customers wherein the merchant point of sale device 130 stores the generated facial template.

In certain other examples, the camera module 132 continuously captures a video feed of its surroundings as users 101 enter and leave the vicinity of one or more merchant POS devices 130 over the course of a certain time period. In this example, the merchant point of sale device 130 and/or camera module 132 is able to continuously monitor the incoming video feed to detect faces from extracted frames of the video feed. In this example, the camera module 132, each time the camera module 132 detects the presence of one or more faces in the video feed, the camera module 132 extracts a frame of the video feed comprising one or more facial images of one or more corresponding detected faces and creates facial templates based on the extracted one or more facial images. In this example, the merchant point of sale device 130 stores facial templates in the log of facial templates of current customers as they are generated. In this example, as the camera module 132 or the merchant point of sale device 130 generates a subsequent facial templates, the merchant point of sale device 130 determines whether the generated subsequent facial template is similar to within a threshold compared to any of the facial templates already stored in the log of facial templates of current customers. If the generated subsequent facial template is similar to within a threshold to any of the facial templates already stored in the log, the merchant point of sale device, after associating the facial template to one or two particular merchant POS devices 130 based on the position of the associated facial images in the extracted frame of the captured video, adds the facial template to the log of facial templates of current customers. If the generated subsequent facial template is not similar to within a threshold to any facial templates already stored in the log of facial templates of current customers, the merchant point of sale device 130 deletes or otherwise ignores and/or does nothing with the generated facial template. In this example, if the merchant point of sale device 130 determines that certain facial image is no longer in the field of the video feed, the corresponding facial template is deleted from the log of facial templates of current customers.

In block 940, the camera module 132 deletes the captured video and the extracted facial image. For example, the camera module 132 does not store captured images or video. In this example, facial templates generated by the camera module 132 comprise computer code representations of facial images of users 101. In this example, after generating a facial template or after a threshold time has passed after capturing video or images or extracting an image from a video, the merchant camera device 140 deletes any captured or extracted video or images.

In block 950, the merchant point of sale device 130 retrieves facial templates from the current customer log. For example, the current customer log comprises facial templates received from the account management system 160 corresponding to all current users 101 whose associated user computing devices 110 are located within a network distance of a merchant beacon device 120.

In block 960, the merchant point of sale device 130 compares the generated facial template from captured facial image to facial templates from the current customer log. The merchant point of sale device 130 may compare each feature of the captured facial template from the current customer log to a corresponding feature in the generated facial template to identify similarities and differences. For example, if one feature is the length of the nose of the user 101, then the stored nose length of the generated facial template is compared to the nose length of the captured facial template. Any suitable comparison of any quantifiable features may be performed.

In block 970, the merchant point of sale device 130 determines whether there is a match between the generated facial template and one of the facial templates from the current customer log. If a facial template from the current customer log matches the generated facial template, the method 260 proceeds to block 270 in FIG. 2. For example, the merchant point of sale device 130 processes a transaction.

If none of the facial templates from the current customer log matches the generated facial template, the method 260 repeats the process to seek a match.

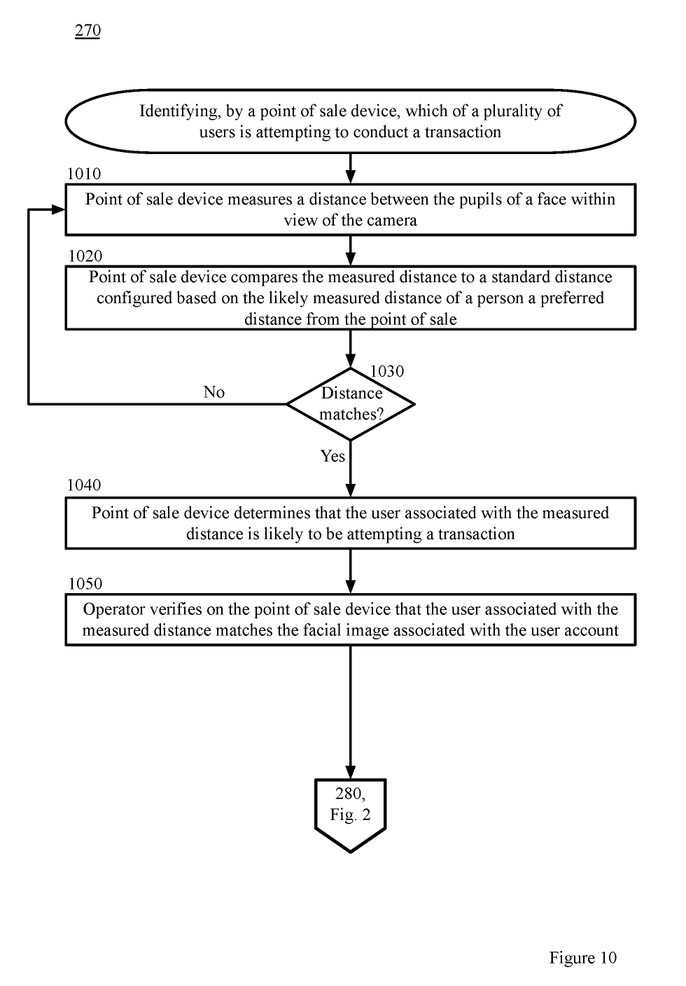

Returning to block 270, in FIG. 2, the point of sale device 130 identifies which of a plurality of users is attempting to conduct a transaction. The method 270 for identifying, by an account management system 160, which of a plurality of users is attempting to conduct a transaction is described in more detail hereinafter with reference to the method 270 described in FIG. 10.

FIG. 10 is a block flow diagram depicting a method 270 for identifying, by an account management system 160, which of a plurality of users is attempting to conduct a transaction, in accordance with certain examples. The method 270 is described with reference to the components illustrated in FIG. 1.

In block 1010, the point of sale device 130 measures a distance between the pupils of a face within view of the camera module 132.

The point of sale device 130, the merchant system, the account management system 160, or any other suitable system may analyze the image to determine which person in the image is likely the person at the front of the line to conduct a transaction. Throughout the specification, the point of sale device 130 will represent any computing system that performs the functions of the method 270. For example, the point of sale device 130 may transmit the facial images to the account management system 160 for analysis and receive the analysis therefrom.

The point of sale device 130 identifies the pupils of one of the faces in the image that was obtained in the method 260 of FIG. 9, or in any other suitable fashion. Any other suitable part of the eye may be utilized instead of the pupils. The point of sale device 130 calculates the distance between the pupils, such as by counting the number of pixels between the pupils. Any other suitable method of determining the distance between the pupils may be used, such as by comparison against a measurement standard, a mathematical manipulation of the digital data of the image, or any other suitable method.

As discussed herein, the pupil distance is only one example measurement that may be used. Any other suitable facial or biometric measurement may be used, such as the length of the nose or the distance between the ears. The distance between the pupils is a particularly useful measurement because this distance is substantially the same across a large percentage of the population, while other facial measurements may have a greater variance across the population.

In block 1020, the point of sale device 130 compares the calculated distance between the pupils to a standard measurement that is configured or calibrated based on the likely measured distance of a user 101 who is standing a preferred distance from the point of sale device 130. For example, to train the point of sale device 130 or to determine a configured standard, an operator 102 may stand in one or more likely positions to be conducting a transaction and have the point of sale device 130 capture an image. Knowing that the operator was in a likely position to make a transaction, the point of sale device 130 measures the distance between the pupils of the operator 102 in the image and stores the measurement. The training procedure may be performed from more than one likely position to allow a measurement tolerance to be predicted. That is, the point of sale device 130 may determine that the standard allows a match for a user 101 in a specific position plus or minus one meter or other desired tolerance in any direction.

Additionally or alternatively, the distance from the camera may be estimated based on the distance between the pupils because the distance between the pupils is substantially consistent across a high percentage of the population, typically approximately 2.2 inches for an average adult. Based on this known distance between pupils, the point of sale device 130 may calculate a distance of a user 101 from the camera by a mathematical calculation of the number of pixels between the pupils of the user 101. The standard is determined based on the distance between the pupils of a typical user 101 in the image when the user 101 is at the preferred distance from the camera. The standard may be calculated mathematically, determined based on trial and error, calibrated, or determined in any other suitable manner. For example, when a user 101 is closer to the camera (and thus filling up a greater percentage of the image), the number of pixels between the pupils of the user 101 will be greater. When a user 101 is farther from the camera (and thus filling up a lesser percentage of the image), the number of pixels between the pupils of the user 101 will be smaller. The number of pixels between the pupils of the user 101 is calibrated to reflect the distance the user 101 is from the camera. For example, a user 101 with 2.2 inches between the pupils may have a measurement of 10 pixels when the user 101 is 20 feet away from the camera. The same user 101 may have a measurement of 100 pixels when the user 101 is 2 feet away from the camera.

In block 1020, the point of sale device 130 compares the measured distance to the standard distance configured based on the likely measured distance of a person positioned a preferred distance from the point of sale 130. In an example, the point of sale device 130 has a stored standard that a user 101 in the likely location to be performing a transaction will have 75-85 pixels between the pupils when captured on a 1080 pixel camera.

The standard may be based on an average number of pixels from previous measurements. The standard may be based on an average number of pixels based on an average distance between the pupils of a group of people, such as a group comprised of all adults. The standard may be based on an average distance between the pupils of adults from a particular geographic region. Any other suitable calculation may be used to determine the average number of pixels for the configured standard. The point of sale device 130 counts the number of pixels on the image of the user 101 for a comparison to the standard. The comparison may be determined to be a match if the number of pixels on the image of the user 101 is within a configured range of the standard number of pixels. That is, if the standard number of pixels is 80 pixels, a match may be determined if the number of pixels on the image of the user 101 is calculated to be within 5% of the 80 pixels, within 5 pixels of the 80 pixels, or within any configured range of 80 pixels.

In another example, the point of sale device 130 determines if the gender of the user 101 is known to allow a more refined standard for the distance between the pupils. That is, male users and female users may have a different average distance between the pupils. The distance may be slightly greater for a male. If the gender of the user 101 is known to be a male, then a standard distance for males may be used in the comparison. Similarly, if the gender of the user 101 is known to be a female, then a standard distance for females may be used in the comparison. Any other known characteristic for the user 101 may be employed to allow a more specific, or accurate, standard to be used.

In block 1030, the point of sale device 130 determines if the comparison of the measured distance matches the standard. If the measured distance does not match the standard, then the point of sale device 130 returns to block 1010 to obtain another image for comparison. In the example, the point of sale device 130 counts 60 pixels between the pupils of the user 101. This would indicate that the user 101 is farther away from the camera than the preferred position of a user 101 attempting a transaction, which would produce a calculation of 75-85 pixels. By returning to block 1010, the point of sale device 130 may identify a second user in the image or obtain a second image of the user 101. Any iteration of the process may be repeated until an image is obtained that indicates that the user 101 is in the preferred position of a user 101 attempting a transaction.

If the distance matches the standard, then block 1030 proceeds to block 1040. For example, the number of pixels between the pupils of the user 101 is counted at 78. This would indicate that the user 101 is in the preferred position. The user 101 is determined to be located at the preferred position before the point of sale device 130.

In block 1040, the point of sale device 130 determines that the user 101 associated with the measured distance is likely attempting a transaction based on the match to the standard. The user 101 is determined to be the user 101 that is attempting the transaction. The counting of pixels described herein is merely an example calculation. Any suitable method, such as a geometric method, a mathematical method, a 3D modeling method, or other method may be utilized. For example, a 3D model may be created of the space before the point of sale device 130 using two or more cameras to map a 3D space.

In an alternate example, the point of sale device 130 performs the analysis of two or more facial images simultaneously. That is, the point of sale device 130 captures multiple facial images and performs the operations of the method 270 on both images simultaneously. The image that produces the match is selected as the likely transactor.

In block 1050, the operator 102 verifies on the POS device 130 that the user 101 associated with the measured distance matches the facial image associated with the user account. For example, the point of sale device 130 may display an image of the user 101 to the POS device operator 102 on a user interface of the POS device 130 to allow the operator 102 to note if the appropriate user 101 is attempting the purchase. The image might be an image captured by the POS device 130 or the image may be an image associated with the user account. If the user 101 does not appear to be the person that is in the image associated with the user account, then the operator 102 may ask for further identification or perform any other suitable actions to verify the user 101. If the user 101 does match the image, then the salesperson may indicate the match in any suitable manner, such as by actuating a virtual button to conduct the transaction.

From block 1050, the method 270 returns to block 280 of FIG. 2.

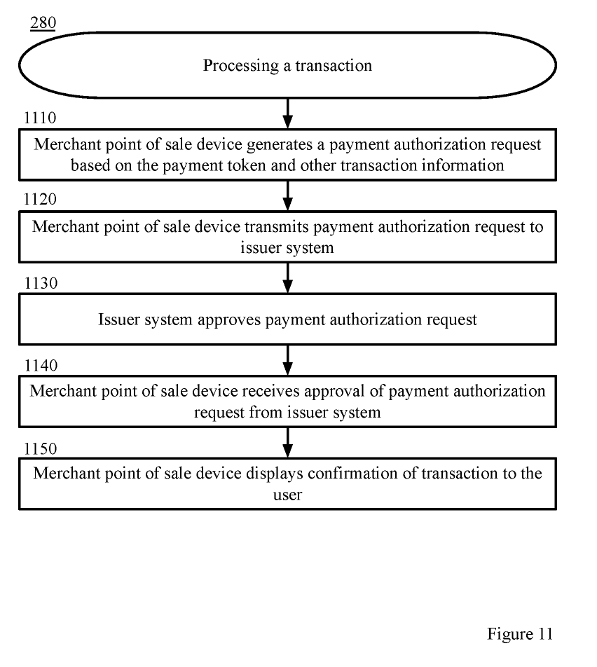

Returning to FIG. 2, in block 280, a transaction is processed. The method for processing a transaction is described in more detail hereinafter with reference to the method 280 described in FIG. 11.

FIG. 11 is a block flow diagram depicting a method 280 for processing a transaction, in accordance with certain examples. The method 280 is described with reference to the components illustrated in FIG. 1.

In block 1110, the merchant point of sale device 130 generates a payment authorization request based on the payment token and other transaction information. In an example, the payment authorization request comprises the payment token received from the account management system 160 for the user 101 along with transaction details including a transaction total, a description of one or more items being purchased, a merchant identifier, a merchant payment account identifier, and/or other relevant transaction details.

In block 1120, the merchant point of sale device 130 transmits the payment authorization request to the issuer system 150. For example, the merchant point of sale device 130 communicates the payment authorization request to the issuer system 150 via the network 105.

In block 1130, the issuer system 150 approves the payment authorization request. In an example, the issuer system 150 identifies the user payment account based on the received payment token. For example, the issuer system 150 accesses a database that associates payment tokens with user 101 payment account identifiers. In an example, the database may further associate payment tokens with one or more conditions, such as a length of time for which the payment token is valid. For example, a payment token may only be valid for a threshold length of time, for example one hour, after it is generated by the account management system 130. In this example, as part of the transaction details in the payment authorization request, a current timestamp is received from the merchant point of sale device 130 and the issuer system 150 compares the received timestamp from the transaction details to the one or more time conditions described in the database associated with the payment token and/or one or more data received from the account management system 160 at the time of the receipt of the payment token.

In another example, the payment token is valid only for use at a particular merchant system. In this example, the transaction details received with the payment authorization request from the merchant point of sale device 130 identifier comprise a merchant system identifier. In this example, the issuer system 150 determines that the payment token is valid if the merchant identifier received in the transaction details of the payment authorization request match the merchant identifier in the one or more conditions associated with the payment token in the database. In certain other examples, other conditions related to time, location, merchant identifier, or a combination of these conditions and/or other conditions may be specified in the database as associated with one or more particular payment tokens. In an example, the issuer system 150 verifies that a payment token received as part of a payment authorization request is valid based at least in part on data received from the merchant point of sale device 130 and/or data currently available to the issuer system 150. In an example, to process the transaction, the issuer system 150 identifies the user payment account associated with the received payment token in the database processes the transaction using the transaction details and the user payment account information.

In certain example, the payment token is associated with a loyalty account and consists of only options to purchase an item with loyalty points, rewards, or offers. In this example, a payment instrument may or may not be associated with the loyalty account. The loyalty account data may serve in lieu of the payment instrument process described herein.

In block 1140, the merchant point of sale device 130 receives an approval of the payment authorization request from the issuer system 150. In an example, the issuer system 150 either approves or declines the payment authorization request. In this example, the issuer system 150 may base the decision of whether to approve or decline the payment authorization request based on a total amount of transaction the current available credit of the user 101 for the user 101 payment account. In an example, the merchant point of sale device 130 receives, via the network 105, the approval of the payment authorization request from the issuer system 150 if the issuer system 150 approves the payment authorization request. In another example, the merchant point of sale device 130 receives a notice of declined payment authorization request from the issuer system 150 via the network 105 if the issuer system 150 declines the payment authorization request.

In block 1150, the merchant point of sale device 130 displays a confirmation of the approved transaction to the user 101. An example confirmation of the approved transaction may include a total amount charged to the user 101 payment account, an identification of the user 101 payment account, a merchant system name, and/or other relevant or useful information. In another example, the merchant point of sale device 130 displays a notification of a declined transaction in response to receiving a notice of declined payment authorization request from the issuer system 150. For example, the merchant point of sale device 130 displays a message reading “This transaction has been declined” to the user via the user interface 135 of the merchant point of sale device 130. In another example, the merchant point of sale device 130 prints a receipt for the user 101.

In certain examples, the transaction is not conducted based on the identification, but a loyalty program is applied to the account of the identified user. For example, if the user 101 conducts the transaction in cash, the point of sale device 130, may communicate the transaction details to the issuer system 150, and the issuer system 150 applies the transaction data to a user loyalty account on the issuer system 150 or managed by a third party loyalty system. For example, the issuer system 150 identifies a user 101 that pays $30 for a transaction in cash. The issuer system 150 notes the identification of the user 101 and identifies a user loyalty account associated with the user 101. The issuer system 150 applies an appropriate number of points to the user loyalty account based on the transaction. In another example, the issuer system 150 applies a reward, offer, or other loyalty benefit to the user loyalty account, such as a new offer to be redeemed at the next transaction.

In another example, a transaction is not conducted based on the identification. Instead, any type of interaction that is based on a determination that person is near a location may be conducted. For example, a user 101 may be granted admission to a secure location, such as an apartment building, based on the determination. In another example, a person may be access to a ticketed location, such as an airline flight, based on the determination. Any other suitable interaction may be prompted based on the methods described herein.

### Other Examples

FIG. 12 depicts a computing machine 2000 and a module 2050 in accordance with certain examples. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a vehicular information system, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

In situations in which the systems discussed here collect personal information about users, or may make use of personal information, the users may be provided with an opportunity or option to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement an embodiment of the disclosed embodiments based on the appended flow charts and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the embodiments presented previously are illustrative, and, in alternative embodiments, certain acts can be performed in a different order, in parallel with one another, omitted entirely, and/or combined between different examples, and/or certain additional acts can be performed, without departing from the scope and spirit of various embodiments. Accordingly, such alternative embodiments are included in the scope of the following claims, which are to be accorded the broadest interpretation so as to encompass such alternate embodiments.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the examples, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

What is claimed is:

1. A computer-implemented method to use facial images to determine that a person is near a location, comprising:
receiving, by the one or more computing devices, a plurality of facial images captured by a camera proximate to the location;
identifying, by the one or more computing devices, the pupils in a first image of the plurality of facial images;
determining, by the one or more computing devices, an image distance between the pupils in the first image;
determining, by the one or more computing devices, that the image distance between the pupils in the first image satisfies a predetermined distance relationship; and
based on determining that the image distance between the pupils satisfies the predetermined distance relationship, providing, by the one or more computing devices, information associated with the first image.

2. The computer-implemented method of claim 1, wherein the image distance is determined by counting the pixels in the facial image between the pupils.

3. The computer-implemented method of claim 1, wherein the predetermined distance relationship is determined based on the distance from the camera to the location.

4. The computer-implemented method of claim 1, wherein the predetermined distance relationship is determined based on one or more of the type of the camera, the image format used by the camera, and the orientation of the facial image.

5. The computer-implemented method of claim 1, wherein the predetermined distance relationship is based on an average distance between the pupils of a cross section of a group of people.

6. The computer-implemented method of claim 1, wherein the match is determined if the distance is within a configured percentage of the configured distance.

7. The computer-implemented method of claim 1, further comprising:
comparing, by the one or more computing devices, the first image to a set of facial templates of current customers determined to near the location;
determining, by the one or more computing devices, that a match exists between the first image and a facial template in the set of facial templates; and
identifying, by the one or more computing devices, a user account based on the matched facial template.

8. The computer-implemented method of claim 7, wherein providing information associated with the first image comprises providing the user account.

9. A computer program product, comprising:
a non-transitory computer-readable medium having computer-executable program instructions embodied thereon that when executed by a computer cause the computer to use facial images to determine that a person is near a location, the computer-executable program instructions comprising:
computer-executable program instructions to receive a plurality of facial images captured by a camera proximate to the location;
computer-executable program instructions to identify the pupils in a first image of the plurality of facial images;
computer-executable program instructions to determine an image distance between the pupils in the first image;
computer-executable program instructions to determine that the image distance between the pupils in the first image satisfies a predetermined distance relationship; and
based on determining that the image distance between the pupils satisfies the predetermined distance relationship, providing information associated with the first image.

10. The computer program product of claim 10, wherein the image distance is determined by counting the pixels in the facial image between the pupils.

11. The computer program product of claim 10, wherein the predetermined distance relationship is determined based on the distance from the camera to the location.

12. The computer program product of claim 10, wherein the predetermined distance relationship is determined based on one or more of the type of the camera, the image format used by the camera, and the orientation of the facial image.

13. The computer program product of claim 10, wherein the predetermined distance relationship is based on an average distance between the pupils of a cross section of a group of people.

14. The computer program product of claim 10, wherein the match is determined if the distance is within a configured percentage of the configured distance.

15. A system to use facial images to determine that a person is near a location, comprising:
a storage device; and
a processor communicatively coupled to the storage device, wherein the processor executes application code instructions that are stored in the storage device to cause the system to:
receive a plurality of facial images captured by a camera proximate to the location;
identify the pupils in a first image of the plurality of facial images;
determine an image distance between the pupils in the first image;
determine that the image distance between the pupils in the first image satisfies a predetermined distance relationship; and
based on determining that the image distance between the pupils satisfies the predetermined distance relationship, providing information associated with the first image.

16. The system of claim 15, wherein the image distance is determined by counting the pixels in the facial image between the pupils.

17. The system of claim 15, further comprising application code instructions that are stored in the storage device to cause the system to:
comparing the first image to a set of facial templates of current customers determined to near the location;
determining that a match exists between the first image and a facial template in the set of facial templates; and
identifying a user account based on the matched facial template.

18. The system of claim 15, wherein providing information associated with the first image comprises providing the user account.

19. The system of claim 15, wherein the image distance is determined by counting the pixels in the facial image between the pupils.

20. The system of claim 15, wherein the predetermined distance relationship is determined based on the distance from the camera to the location.
